(19) United States
(12) Patent Application Publication
(10) Pub. No.: US 2025/0336494 A1
(43) Pub. Date: Oct. 30, 2025

(54) TAIWA HUMAN TRANSFORMATION SYSTEM AND METHOD

(71) Applicant: Oliver Phillips, Santa Barbara, CA (US)

(72) Inventor: Oliver Phillips, Santa Barbara, CA (US)

(73) Assignee: Empowerment Holdings LLC, Santa Barbara, CA (US)

(21) Appl. No.: 19/189,096

(22) Filed: Apr. 24, 2025

Related U.S. Application Data
(60) Provisional application No. 63/640,203, filed on Apr. 30, 2024.

(51) Int. Cl. G16H 20/00; G06N 20/00

(52) U.S. Cl. CPC G16H 20/00; G06N 20/00

(57) ABSTRACT

A system for continuous quality of life improvement, vocational progress, and human transformation is provided. The system comprises a computer and an application executing thereon that receives input describing a user. The input comprises material received via a dialog system comprising scraped online data describing the user and comprises data received from wearables attached to the user. The input further comprises personal and professional goals of the user and is provided for inclusion in a human transformation program. The system accesses stored records containing content at least similar to the received input, the stored records associated with previous users partaking in the program. The system also applies predictive analytics to the input in combination with selected contents of the stored records to generate courses of behavior development for the user. The system also provides selected educational and transformational microprocesses based on the generated courses.

### CROSS REFERENCE WITH RELATED APPLICATIONS

The present non-provisional patent application is related to U.S. Provisional Patent Application No. 63/640,203 filed Apr. 30, 2024, the contents of which are incorporated herein in their entirety.

### FIELD OF THE DISCLOSURE

The present disclosure is in the field of development of transformative programs. More particularly, the present disclosure provides systems and methods of transforming received participant input comprising present life situation, states, traits and goals into programs for participant use in working toward substantial transformation toward achievement of personal and professional goals.

### BACKGROUND

Human transformation is an intentional, ongoing process of developing your full potential across different aspects of your life. It encompasses personal growth, spiritual evolution, and skill development, leading to a more fulfilling and meaningful existence.

Human transformation may refer to a profound and often dramatic change in a person's character, beliefs, or way of life, often leading to a more positive or fulfilling existence. Personal transformation involves a conscious effort to change oneself, becoming the person he or she wants to be or aligning with their inner values. It can encompass areas like personal growth, overcoming challenges, and developing new skills or habits.

In a business or organizational context, human transformation refers to the process of change that individuals and teams undergo as a company evolves its strategies, processes, or technologies. This involves a shift in mindset, behavior, and skills to better align with the organization's goals and the evolving business landscape. With spiritual transformation, some perspectives, like those found in certain religious or spiritual traditions, view transformation as a journey towards a higher state of being or closer to a divine entity.

Radically human transformation emphasizes empowering people to lead, operate, think, decide, and adapt in rapidly changing environments, fostering a mindset shift that enables ongoing reinvention. Human-centric transformation focuses on involving people in modifying long-term behaviors and core beliefs to achieve a desired result, emphasizing transparency, listening to people's needs, and fostering innovation.

### BRIEF DESCRIPTION OF THE FIGURE

FIG. 1 is a block diagram of a system of human transformation according to an embodiment of the present disclosure.

### DETAILED DESCRIPTION

Systems and methods described provide parties with comprehensive guidance through transformative processes of personal and professional growth and development. Systems receive and analyze input from parties about their present situations and conditions including health, financial situation, and social interactions as well as thoughts and attitudes. Personal goals are also received. The system applies artificial intelligence (AI) including generative pre-trained transformers (GPT) and large language models (LLM) to the submitted material. It integrates the AI results with stored knowledge about the experiences of previous users of similar programs.

The system generates a program including training and exercises for the particular user and later stores and processes the user's feedback about his/her experiences with the program. The system is directed to leading the user to welcome guidance through a transformative process of personal and professional growth.

The Al-based system uses predictive analytics based on available research and proprietary user data processing. It provides various alternative pathways of personal and professional development including the most probable and the most possible courses of behavior development along a spectrum of positive traits leading the user to success in their stated goals.

The system effectively “learns” from the process of integrating newly received feedback and other information with stored records of previous' users experiences with similar programs they embarked upon and completed. The system analyzes the current input in light of stored material about prior users. Conversely, the system may make adjustments to stored records and AI components based on new lessons learned from new users. The system improves itself iteratively with each new user serviced. The AI-driven processing integrates newly received user input with stored material to improve the quality and precision of training and exercises given to future users.

Previous users whose stored historical input about the their experiences is used in new programs that turn out to be successful may receive remuneration. Such remuneration may be in the form of tokens that may be used to purchase new services from the system. Tokens may be freely exchanged in an open market manner.

A user's present outcomes, states and traits are observed and compared with the user's stated goals. Outcomes include observable health, wealth and social interactions and integration across those areas. States define the user's current mental, emotional, physical, and spiritual wellbeing.

Traits define the user's mental, emotional, imaginative, verbal, and physical recurring behaviors. Traits may be described as thoughts, attitudes, imagination, words, and actions and may be represented by the acronym TAIWA®.

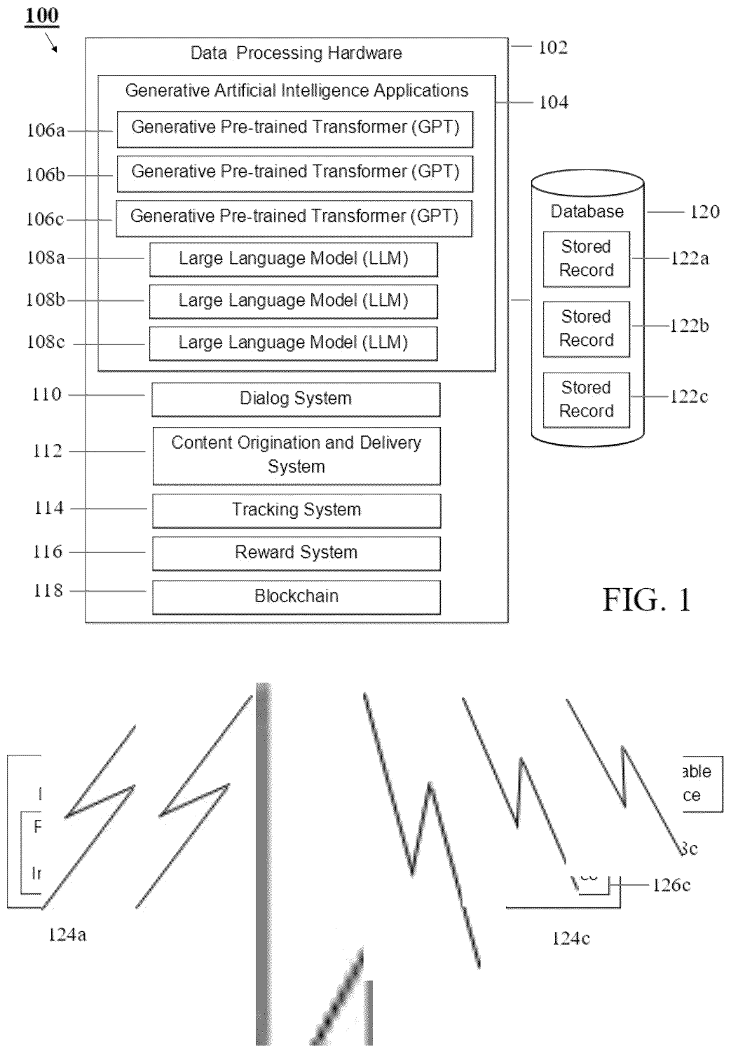

Turning to the figures, FIG. 1 is a block diagram of a system of human transformation according to an embodiment of the present disclosure. FIG. 1 depicts components and interactions of a system 100 of human transformation. System 100 comprises data processing hardware 102 comprising many physical computing, storage, and communications hardware and software components including generative artificial intelligence applications 104. For purposes of brevity, the data processing hardware 102 and the generative artificial intelligence applications 104 may be referred to as hardware 102 and applications 104 respectively.

The hardware 102 may comprise one or multiple computers and other devices that may be physically situated at a single or multiple geographic locations. In many embodiments, the hardware 102 comprises specialized, non-generic computing devices.

The applications 104 comprise generative pre-trained transformers 106a-c, also referred to a GPT 106a-c, and large language models 108a-c, also referred to as LLM 108a-c. System 100 also comprises a dialog system 110, a content origination and delivery system 112, a tracking system 114, a reward system 116, and a blockchain 118. While these five components 110-118 are depicted as components of the hardware 102 and executing thereon and not part of the applications 104, in various embodiments these components may be entirely software, entirely hardware, or a portion of each. Further, portions of these five components may not execute on the hardware 102 and may execute partially elsewhere. Also, portions of these five components may be part of the applications 104 and execute based on instructions from the applications 104.

System 100 also comprises a database 120 and stored records 122a-c. System 100 also comprises user devices 124a-c, platform user interfaces 126a-c, and wearable devices 128a-c. The platform user interfaces 126a-c execute on the user devices 124a-c and enable a user to interact with the system. This may include submitting information including the initial input, receiving transformation program material, and submitting feedback.

Wearable devices 128a-c may include glucose monitors, wearable ECG monitors, blood pressure monitors, pulse oximeters, smartwatches with health features, temperature tracking wearable devices, sleep tracking devices, fitness tracking devices, smart rings, and smart clothing for health monitoring. Wearable devices 128a-c may communicate wirelessly with the hardware 102 or may be submit material through the user devices 124a-c which then transmit to the hardware 102.

While system 100 provides and FIG. 1 depicts quantity three of GPT 106a-c, LLM 108a-c, stored records 122a-c, user devices 124a-c, platform user interfaces 126a-c, and wearable devices 128a-c, this is done for illustration and discussion purposes only. In embodiments, more than or less than quantity three of each of these components may be provided by the system 100.

Systems and methods provided herein may be implemented in phases. While seven phases are described below, system 100 provides for other phases to be added.

### Phase 1: Assessment

A new user logs in and engages through the dialog system 110 that may comprise chat, audio, or video with the artificial intelligence applications 104. Using user dialog, scraped online user data, and hardware data collected though wearables, the system performs user intake and assessment. During intake the system 100 gathers information about the user's current personal and professional real time experience.

The real-world outcomes, states and traits describing the user's present situation and condition may be referred to as REALITY1. The user gains insight from the assessment about his/her current developmental progress in terms of their personal and professional life, their ongoing activities, and results.

### Phase 2: Insight

The user accesses the AI applications 104 and the dialog system 110 and defines their desired, wanted, or needed reality. This may be named REALITY 2 and may be desired, wanted, or needed outcomes, states, and traits as described above.

### Phase 3: Forecast/Choice

System 100 uses predictive analytics based on available research and proprietary user data processing. It provides various alternative pathways of personal and professional development.

### Phase 4: Coaching/Training/Application

System 100 presents the user with selected educational and transformational microprocesses in the forms of exercise and training modules. The user engages in the exercises and training of their selected behavior changes.

Program modules may be provided to the user via at least basic chat, audio, image, video, augmented reality (AR), virtual reality (VR), wearables, and headsets. Data describing the user's developmental choices, efforts and progress are tracked and recorded anonymously. Data from the entire process may be recorded on a blockchain.

### Phase 5: Training Data Tracking

User data is generated by the user engaging in and accomplishing exercises and training and applying the learning and development in “flight mode” and in the real world. User data is generated along all phases of the system. User data is used to improve the AI system to provide better, even more precise and personalized AI coaching and training to the user and future users.

The user's initial state, their chosen path to change and transformation, and their specific efforts in accomplishing the educational and transformational microprocesses may all be recorded on the blockchain. The user's application of their development in “flight mode” and in the real world, and the actual results of these efforts in their lives may also recorded on the blockchain and become part of the AI-coaching data repository.

### Phase 6: Monetization

Users' efforts to engage and develop themselves from their present situation to desired reality via application of AI coaching-directed educational and transformational training microprocesses, the specific actions taken by users, the AI-coaching modules completed, and all the results achieved are processed and recorded on the blockchain.

This data set represents the user's actual personal transformational journey and therefore may represent the user's contribution to the overall value of the entire system. This contribution of data may be remunerated according to actual demand.

When a new user begins using the system, their AI-coaching journey will be informed by and benefit from prior users' accumulated data in transforming from their own REALITY 1 into REALITY 2. When a new user accesses a particular transformational journey on the platform, previous users whose data has contributed to the clarification and optimization of this individual's transformative journey by previously providing their own user data on the blockchain may receive rewards for that contribution in the form of TAIWA® tokens or other items of value.

The dispersing of the TAIWA® tokens may be fractional and automatic and is managed by the system. The value of the tokens is established through basic principles of supply and demand. Tokens can be converted on the TAIWAR platform into new licensed platform access and usage or traded off the platform through a digital wallet integration and exchanged on the open market.

### Phase 7: Profile Development

User data and transformational journeys are recorded in TAIWA® digital profiles created for each user. The digital profile is a decentralized ID that is portable. The user may develop a digital asset portfolio consisting of their transformational analyses, exercise and training efforts, experiences, and results data.

In various embodiments, the system may continuously collect and analyze publicly available digital footprint data reflecting user progress. This may comprise material from professional profiles, social media accounts, online publications, digital certifications, and other observable activities relevant to a user's personal and professional transformation journey. Such external data may supplement internally collected behavioral and engagement metrics to enhance AI-driven program optimization and user progress tracking.

The system is also based in part on an “economic flywheel” model and proof of development concept. The system operates as an investment infrastructure wherein users' authentic personal and professional developmental progress becomes a recorded, verified digital asset. Users who engage in self-improvement and demonstrate authentic transformational achievements contribute to an evolving dataset used to optimize programs for future participants. Future user fees and participation generate economic flows that remunerate past contributors, enabling users to earn by investing effort into their own personal and professional growth.

Each user's recorded and verified transformational journey as supported by the blockchain serves as immutable proof of their efforts toward personal and professional development. This proof becomes a digital asset eligible for remuneration based on its contribution to optimizing future user programs, as well as through other monetization pathways including licensing, digital credentialing, decentralized identity assets, or open marketplace trading.

Thus, the activity of striving toward one's goals and realizing one's potential across all areas of development becomes a monetizable economic activity, creating a self-reinforcing system where individual progress catalyzes collective system improvement and equitable value sharing. Users are hence incentivized to strive for progress and do well as their recorded experiences, accomplishments, and knowledge gained boost their value in the system provided herein. In some embodiments, alternative revenue models may also be utilized.

User feedback as used herein is to be interpreted broadly and may encompass significantly more than what the user might explicitly say or write. Any user actions or behavior, whether measured, observed, and reported by the user him/herself or by other parties or devices may also be considered user feedback for purposes of determining the user's progress toward goals. While the user may submit materials describing actions and results, independent observations of the user's behavior may also be considered user feedback.

Similar to a 360 review in a workplace, feedback may be gathered from multiple sources, including managers, peers, and other associates to develop a comprehensive perspective on a user's performance and behaviors. As with user feedback, user input is not limited to input entered exclusively by the user. User input may comprise scraped external data gathered both at initial assessment and as well as during ongoing program progress tracking.

Systems and methods intend that data collection is broad and covers many sources of information about a user and his/her progress under a program. Data collection is both explicit and passive and is gathered from wearable and other devices. It also comprises external internet footprint data. Data integrity is supported by storage on blockchains as described above.

Training methods provided herein, which are heavily AI-based and are to be protected intellectual property, are future-proofed. Such methods include Reinforcement Learning from Human Feedback or RLHF, a machine learning technique where an AI model (often a Large Language Model) learns through feedback from human annotators, guiding its behavior and improving its ability to generate more helpful, aligned, and safe outputs. Emerging synthetic and self-generated AI training improvements are also valuable and protected.

System security is strong and is protected by blockchain which is reliable and immutable. Security of data is critical for trust and regulatory acceptance and compliance.

Systems and methods are also directed to maximizing licensing value across the portfolio. Based on many years of building valuable intelligence from user experiences, the portfolio will contain material that may be licensed to other organizations seeking to build character among its people. These organizations may include corporations, governments and universities. Also, publishers, webinar and seminar producers, and motivational speakers and material developers may obtain the valuable material via licensing.

Systems and methods also may be employed in embodiments to develop defensive intellectual property to protect users of products developed herein from becoming the targets of litigation or harassment from parties believing they have a legal standing to pursue such action. Further, intellectual property assets developed using systems and methods provided herein may have strategic value when synergized and integrated into a portfolio that encompasses various programs, approaches, and stored user success stories. Sectors that may use the assets may include human resources, healthcare, fitness, education, and wellness. As discussed above, the economic flywheel model allows users' developmental progress to become assets eligible for multiple forms of monetization.

In an embodiment, a system for continuous quality of life improvement, vocational progress, and human transformation is provided. The system comprises a computer and an application executing thereon that receives input describing a user. The input comprises material received via a dialog system comprising scraped online data describing the user and comprises data received from wearables attached to the user. The input further comprises personal and professional goals of the user and is provided for inclusion in a human transformation program. The system accesses stored records containing content at least similar to the received input, the stored records associated with previous users partaking in the program. The system also applies predictive analytics to the input in combination with selected contents of the stored records to generate courses of behavior development for the user. The system also provides selected educational and transformational microprocesses based on the generated courses.

The input comprises real world outcomes comprising the user's present health, wealth, and social interactions, wherein the input further comprises states comprising the user's current mental, emotional, physical, and spiritual wellbeing, and wherein the input further comprises traits comprising the user's mental, emotional, imaginative, verbal, and physical recurring behaviors. The stored records further comprise outcomes of participation in the program by previous users, the application of the predictive analytics incorporating at least portions of the outcomes.

The microprocesses comprise exercises and training modules to produce behavior changes of the user directed to reaching of the goals. The system operates on generative artificial intelligence comprising large language models and generative pre-training transformers.

The system uses predictive artificial intelligence to identify probability and optimization. The received input, program provided, and user efforts directed to educational and transformational microprocesses are recorded on a blockchain.

Modules associated with the microprocess are delivered by the system via at least one of chat, audio, image, video, augmented reality, virtual reality, wearables, and headsets. User data transmission between wearable devices, user devices, and the system is encrypted using end-to-end encryption, and blockchain records incorporate cryptographic hashes to ensure data integrity and tamper resistance. Microprocesses are dynamically personalized based on user persona archetypes generated via clustering algorithms analyzing user states, traits, and goals.

In another embodiment, a method of continually improving programs for human transformation is provided. The method comprises a computer receiving feedback from a user device describing results of a recommended program of personal and professional growth and development undergone by a first user associated with the device, the program developed by the computer. The method also comprises the computer accessing records describing results of previous users, the results associated with similar recommended programs undergone by the previous users. The method also comprises the computer integrating the feedback into the stored records, the integration comprising at least adjusting artificial intelligence components executing on the computer to effectively learn from the feedback and refine the stored records with selected portions of the feedback.

The computer continually integrates the user feedback into stored records and artificial intelligence components. The computer integrating the feedback is directed to developing improved quality of programs for future users.

The method also comprises the computer incorporating received input from the first user in developing the program, the input comprising material received via a dialog system, comprising scraped online data describing the user, and comprising data received from wearables attached to the user, the input further comprising personal and professional goals of the user. The method also comprises the computer processing the input in combination with previously integrated user feedback in the stored record to develop a customized program for the user in view of the goals.

The method also comprises the computer applying predictive analytics to the input in combination with selected contents of the stored records to generate courses of behavior development for the user. Integration of user feedback into artificial intelligence components comprises applying adaptive model fine-tuning methodologies, including reinforcement learning with human feedback (RLHF), synthetic feedback generation, or other machine-driven optimization techniques to improve behavior prediction accuracy.

The feedback from the user device comprises at least one of: (a) explicit user-provided evaluations; and (b) implicit behavioral data comprising user interactions with platform content, completion of exercises, wearable device outputs, and engagement metrics. User progress is further tracked via scraping, collecting, and analyzing publicly available digital footprint data of the user, including online publications, professional profiles, social media activity, and other accessible online records demonstrating changes aligned with program goals.

In yet another embodiment, a system of compensating previous transformative program participants for contributions toward development of new programs is provided. The system comprises an artificial intelligence system that receives a message from a client device describing results associated with a transformative program undertaken by a current client associated with the device. The system also accesses stored material used to assist in developing the program. The system also determines identities of previous clients associated with the stored material. The system also applies artificial intelligence methodologies to the stored records to determine extent to which material contained in the records contributed to the program and a transformative journey associated therewith. The system also remunerates previous individual clients with tokens in accordance with the determined extent of the individual client's recorded transformative journey contributed to the transformative program undertaken by the current client.

The system is directed to compensating previous program users whose stored records of success contributed to development of aspects of the transformative program deemed successful for the current client. The tokens are used to acquire additional services from the system.

The tokens are eligible for open exchange through a digital wallet integration. The system operates on generative artificial intelligence comprising large language models and generative pre-training transformers.

Programs undertaken by the current client and previous clients comprise exercises and training modules to produce behavior changes directed to reaching stated goals. Remuneration provided to prior program participants derives from a portion of revenue or value created by fees paid by subsequent users accessing optimized programs based on recorded developmental journeys.

What is claimed is:

1. A system for continuous quality of life improvement, vocational progress, and human transformation, comprising:
a computer; and
an application executing thereon that:
receives input describing a user, the input comprising material received via a dialog system, comprising scraped online data describing the user, and comprising data received from wearables attached to the user, the input further comprising personal and professional goals of the user, the input received for inclusion in a human transformation program,
accesses stored records containing content at least similar to the received input, the stored records associated with previous users partaking in the program,
applies predictive analytics to the input in combination with selected contents of the stored records to generate courses of behavior development for the user, and
provides selected educational and transformational microprocesses based on the generated courses.

2. The system of claim 1, wherein the input comprises real world outcomes comprising the user's present health, wealth, and social interactions, wherein the input further comprises states comprising the user's current mental, emotional, physical, and spiritual wellbeing, and wherein the input further comprises traits comprising the user's mental, emotional, imaginative, verbal, and physical recurring behaviors.

3. The system of claim 1, wherein the stored records further comprise outcomes of participation in the program by previous users, the application of the predictive analytics incorporating at least portions of the outcomes.

4. The system of claim 1, wherein the microprocesses comprise exercises and training modules to produce behavior changes of the user directed to reaching of the goals.

5. The system of claim 1, wherein the system operates on generative artificial intelligence comprising large language models and generative pre-training transformers.

6. The system of claim 5, wherein the system uses predictive artificial intelligence to identify probability and optimization.

7. The system of claim 1, wherein the received input, program provided, and user efforts directed to educational and transformational microprocesses are recorded on a blockchain.

8. The system of claim 1, wherein modules associated with the microprocesses are delivered by the system via at least one of chat, audio, image, video, augmented reality, virtual reality, wearables, and headsets.

9. The system of claim 1, wherein user data transmission between wearable devices, user devices, and the system is encrypted using end-to-end encryption, and blockchain records incorporate cryptographic hashes to ensure data integrity and tamper resistance.

10. The system of claim 1, wherein microprocesses are dynamically personalized based on user persona archetypes generated via clustering algorithms analyzing user states, traits, and goals.

11. A method of continually improving programs for human transformation, comprising:
a computer receiving feedback from a user device describing results of a recommended program of personal and professional growth and development undergone by a first user associated with the device, the program developed by the computer;
the computer accessing records describing results of previous users, the results associated with similar recommended programs undergone by the previous users;
the computer integrating the feedback into the stored records, the integration comprising at least adjusting artificial intelligence components executing on the computer to effectively learn from the feedback and refine the stored records with selected portions of the feedback.

12. The method of claim 11, wherein the computer continually integrates the user feedback into stored records and artificial intelligence components.

13. The method of claim 12, wherein the computer integrating the feedback is directed to developing improved quality of programs for future users.

14. The method of claim 11, further comprising the computer incorporating received input from the first user in developing the program, the input comprising material received via a dialog system, comprising scraped online data describing the user, and comprising data received from wearables attached to the user, the input further comprising personal and professional goals of the user.

15. The method of claim 11, further comprising the computer processing the input in combination with previously integrated user feedback in the stored record to develop a customized program for the user in view of the goals.

16. The method of claim 11, further comprising the computer applying predictive analytics to the input in combination with selected contents of the stored records to generate courses of behavior development for the user.

17. The method of claim 11, wherein integration of user feedback into artificial intelligence components comprises applying adaptive model fine-tuning methodologies, including reinforcement learning with human feedback (RLHF), synthetic feedback generation, or other machine-driven optimization techniques to improve behavior prediction accuracy.

18. The method of claim 11, wherein the feedback from the user device comprises at least one of: (a) explicit user-provided evaluations, and (b) implicit behavioral data comprising user interactions with platform content, completion of exercises, wearable device outputs, and engagement metrics.

19. The method of claim 11, wherein user progress is further tracked via scraping, collecting, and analyzing publicly available digital footprint data of the user, including online publications, professional profiles, social media activity, and other accessible online records demonstrating changes aligned with program goals.

20. A system of compensating previous transformative program participants for contributions toward development of new programs, comprising:
an artificial intelligence system that:
receives a message from a client device describing results associated with a transformative program undertaken by a current client associated with the device,
accesses stored material used to assist in developing the program,
determines identities of previous clients associated with the stored material,
applies artificial intelligence methodologies to the stored records to determine extent to which material contained in the records contributed to the program and a transformative journey associated therewith, and
remunerates previous individual clients with tokens in accordance with the determined extent of the individual client's recorded transformative journey contributed to the transformative program undertaken by the current client.

21. The system of claim 20, wherein the system is directed to compensating previous program users whose stored records of success contributed to development of aspects of the transformative program deemed successful for the current client.

22. The system of claim 20, wherein the tokens are used to acquire additional services from the system.

23. The system of claim 20, wherein the tokens are eligible for open exchange through a digital wallet integration.

24. The system of claim 20, wherein the system operates on generative artificial intelligence comprising large language models and generative pre-training transformers.

25. The system of claim 20, wherein programs undertaken by the current client and previous clients comprise exercises and training modules to produce behavior changes directed to reaching stated goals.

26. The system of claim 20, wherein remuneration provided to prior program participants derives from a portion of revenue or value created by fees paid by subsequent users accessing optimized programs based on recorded developmental journeys.
